(19) United States
(12) Patent Application Publication
(10) Pub. No.: US 2025/0166761 A1
(43) Pub. Date: May 22, 2025

(54) System and Method for Providing Personal, Medical, and Legal Identifying Information to Medical Professionals

(71) Applicant: Robert Valentin Salinas, Dallas, TX (US)

(72) Inventor: Robert Valentin Salinas, Dallas, TX (US)

(21) Appl. No.: 18/516,874

(22) Filed: Nov. 21, 2023

(51) Int. Cl. G16H 10/65; G06K 7/14

(52) U.S. Cl. CPC G16H 10/65; G06K 7/1417

(57) ABSTRACT

The present invention is a piece of jewelry worn by a holder of a unique Quick Response (QR) code with the QR code permanently inscribed onto the piece of jewelry. The QR code will be linked to a mobile application (app) and website, specifically a unique app page and Uniform Resource Locator (URL) page. The unique pages can be accessed by the holder/wearer through a user portal to create a profile on the unique pages, to input personal, medical, and legal information as well as uploading legal and non-legal documents to their profile. Medical professionals will have access to the general app and website through a user portal. After scanning the holder/wearer's QR code they will have access to the holder/wearer's information; allowing the medical professional to treat the holder/wearer more efficiently.

### TECHNICAL FIELD

This invention relates to Quick Response (QR) Codes; Mobile Applications (Apps); Jewelry.

### BACKGROUND ART

QR codes; Mobile Applications; Jewelry.

### Disclosure of Invention

The present invention is a piece of jewelry worn by a holder of a unique Quick Response (QR) code with the QR code permanently inscribed onto the piece of jewelry. The present invention includes temporary pieces of jewelry such as temporary paper or plastic wristbands. The unique QR code can also be directly stored on the holder's mobile device. The QR code can be scanned by either a QR scanner or a mobile device to gain access to the holder/wearer's personal, medical, and legal information.

Currently, there are medical alert bracelets that alert medical professional to certain medical conditions the wearer has, but none that have a scannable QR code that will give the medical professional much more information about the holder/wearer. As well, currently, there are temporary wristbands with traditional bar codes used at hospitals to get medical information about the wearer, but none are done with QR codes that will allow the medical professional to pull up such a breadth of the holder/wearer's information directly on either their mobile device or the hospital's computer system.

An individualized, unique QR code will be generated and assigned to the wearer of the jewelry, permanently inscribed on the jewelry, and linked to a mobile application (app) and website; specifically a password protected unique app page, within the app, and unique Uniform Resource Locator (URL) page, within the website, accessible through a password protected user portal. Facial recognition can also be utilized in lieu of a password. The holder/wearer will access the unique app page and unique URL page though the password protected user portal to create a personal profile by inputting personal, medical, and legal information about themselves, as well as uploading legal and non-legal documents. Such information includes but is not limited to name, date of birth, address, phone number, email, known allergies, medications, emergency contacts, pre-existing medical conditions, medical insurance information, and the uploading of legal and non-legal documents such as Do Not Resuscitate Orders, Medical Power of Attorneys, Durable Power of Attorneys, Medical Insurance Documents, Medical Records, etc.

Currently, on the market and available to the public are QR codes and QR scanners, but none linked to a mobile application and website that gives medical professionals who scan the QR code such a breadth of information about the holder/wearer of the QR code, that will help treat the holder/wearer more efficiently. Additionally, currently on the market and available to the public there is jewelry, but none with a permanently inscribed QR code that informs medical professionals of the holder/wearer's personal, medical, and legal information and access to the holder/wearer's legal and non-legal documents.

The present invention is a piece of jewelry worn by a holder of a unique QR code with the QR code permanently inscribed onto the piece of jewelry. The jewelry can be for temporary use, such as a plastic or paper wristband. The unique QR code can also be stored on the unique QR code holder's mobile device. The jewelry can be scanned by a QR scanner or mobile device. The QR code will be linked to an app and website, specifically a unique app page and unique URL page accessible only by the holder/wearer of the unique QR code through a password protected user portal. Facial recognition can also be utilized in lieu of a password. The user portal will allow the holder/wearer to create a personal profile and input personal, medical, and legal information, as well as upload legal and non-legal documents about themselves. This information will allow the medical professionals who treat the holder/wearer to obtain much needed personal, medical, and legal information about the holder/wearer in order to treat them more efficiently.

Medical professionals will have access to the general app and website (but not the unique app page or URL page of the holder/wearer of the unique QR code) using a password protected user portal to access the general mobile application and website. Facial recognition can also be utilized in lieu of a password. The medical professional will access the holder/wearer's unique app page and URL page, without the use of an additional password, by scanning the holder/wearer's unique QR code. This allows the medical professional to access the holder/wearer's personal, medical, and legal information, and no one else's. The medical professional's name and information will be displayed on the holder/wearer's profile as well as any optional medical information such as notes and test results prepared by the medical professional. This allows the next medical professional treating the holder/wearer to be aware of who has previously treated the holder/wearer and the medical attention being received by the holder/wearer.

While the invention has been described with reference to the preferred embodiment thereof, it will be appreciated by those of ordinary skill in the art that modifications can be made to the structure and elements of the invention without departing from the spirit and scope of the invention as a whole.

### BRIEF DESCRIPTION OF DRAWINGS

FIG. 1 is a piece of jewelry, in this case a bracelet wristband (labeled A) with a QR code permanently inscribed on the jewelry (labeled B).

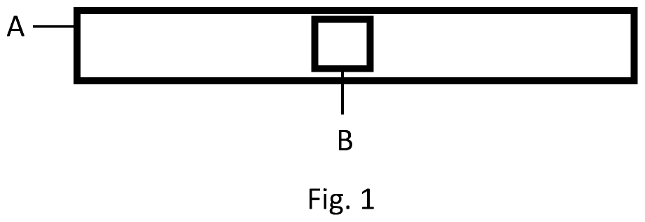

### BEST MODE FOR CARRYING OUT THE INVENTION

A piece of jewelry worn by a holder of a unique QR code with the QR code permanently inscribed onto the piece of jewelry. The QR code will be linked to an app and website, specifically a password protected unique app page and unique URL page. The unique app page and unique URL page can be accessed by the holder/wearer through a password protected user portal to create a personal profile on the unique app page and unique URL page, to input personal, medical, and legal information as well as uploading legal and non-legal documents to their profile. Medical professionals will have access to the general mobile application and website through a password protected user portal. After scanning the holder/wearer's QR code they will have access to the holder/wearer's personal, medical, and legal information without having to input any additional passwords.

The invention claimed is:

1. A piece of jewelry comprising:
a Quick Response (QR) code permanently inscribed on the jewelry;
the QR code linked to a mobile application (app) and website, specifically a unique app page on the app, and unique Uniform Resource Locator (URL) page on the website;
the unique app page and unique URL page containing a personal profile of the jewelry wearer's personal, medical, and legal information.

2. A temporary wristband comprising:
a Quick Response (QR) code permanently inscribed on the temporary wristband;
the QR code linked to a mobile application (app) and website, specifically a unique app page on the app, and unique Uniform Resource Locator (URL) page on the website;
the unique app page and unique URL page containing a personal profile of the temporary wristband wearer's personal, medical, and legal information.

3. A Quick Response (QR) code comprising:
the QR code linked to a mobile application (app) and website, specifically a unique app page on the app, and unique Uniform Resource Locator (URL) page on the website;
the unique app page and unique URL page containing a personal profile of the QR holder's personal, medical, and legal information.
